(19) United States
(12) Patent Application Publication
(10) Pub. No.: US 2013/0172926 A1
(43) Pub. Date: Jul. 4, 2013

(54) GRADUATED COMPRESSION HOSIERY

(71) Applicant: Stephen George Edward Barker, Surrey (GB)

(72) Inventor: Stephen George Edward Barker, Surrey (GB)

(73) Assignee: STUFF OF LIFE LIMITED, EAST SUSSEX (GB)

(21) Appl. No.: 13/806,928

(22) PCT Filed: Jun. 29, 2011
(86) PCT No.: PCT/GB2011/051228

§ 371 Date: Mar. 19, 2013

(30) Foreign Application Priority Data
Jun. 30, 2010 (GB) 1010971.8
Mar. 10, 2011 (GB) 1104088.8

(51) Int. Cl. A61F 13/00

(52) U.S. Cl. CPC A61F 13/00
USPC 606/201

(57) ABSTRACT

An item of graduated compression hosiery additionally comprises a compression panel adapted to provide targeted compression of one or more of the sites for perforating vein communication between the superficial and deeper vein systems in the lower leg.

### FIELD OF THE INVENTION

This invention relates to graduated compression hosiery.

### BACKGROUND OF THE INVENTION

Graduated compression (GC) has long been a mainstay of mechanical prevention of venous thromboembolism (VTE) and as a means to help prevent its sequelae, often described as the post-thrombotic syndrome (PTS). In addition, GC has known benefits for managing varicose veins and for everyday wear in terms of prevention of tired, swollen, uncomfortable legs towards the end of the working day, with travel, with pregnancy and in sports recovery. Many of these functions are associated with enhancement of the ‘calf pump’, otherwise known, colloquially, as the body's second heart.

The ‘calf pump’ represents the action of calf muscles squeezing against the resistive deep fascia to push venous blood back towards the heart, against gravity. To aid in this process, the deep veins have uni-directional valves that prevent venous reflux. Superficial veins (those that are just below the skin surface, above the fascial layer, and that can become varicose) are connected to the deep veins via a system of (usually anatomically fairly standard) perforating (or communicating) veins, that allow uni-directional flow also, from superficial to deep systems.

VTE represents an abnormal event whereby blood clots within the (more often) deep veins. There are many recognised causes, including post-mechanical damage to the veins, e.g. with trauma, post-surgery (and in particular, orthopaedic surgery), blood coagulopathies, long haul flight and previous VTE. The effects of a blood clot forming in the deep veins, i.e. a deep vein thrombosis (DVT), are both immediate and long-term, and it is the latter that is associated with PTS.

Treatment of VTE is usually a combination of long-term anti-coagulation therapy (often with warfarin) and higher compression GC hosiery. The aim is to minimise recurrent VTE, improve venous return and to minimise the chances of PTS and, later, possible limb ulceration.

GC also has a role in the management of varicose veins, both as a treatment in its own right for those who do not want any form of surgical intervention, and as a simple means to keep the limb more comfortable throughout the day.

General ‘well-being’ issues can also be addressed by improvement to normal ‘calf pump’ function, by use of GC hosiery as everyday wear, for maternity use and for sports recovery and perhaps also, for sports performance enhancement. GC hosiery is of known benefit to shop workers, for example, standing on their feet all day, in the prevention of calf aching and swelling. In the second and third trimesters, GC hosiery can help minimise calf ache and lower limb swelling, common with pregnancy. In sport, GC hosiery can improve recovery times between events and be considered to improve ‘performance’ by aiding venous return and perhaps, improve measures such as the anaerobic threshold.

GC hosiery has changed little for many years. All available products are based on an (essentially) agreed classification for graduated compression, giving rise to Classes I, II, III and IV compression, with products varying also by length (e.g. below knee, mid-thigh, full length (leg) or as full tights), composition (e.g. all synthetic yarns, such as mostly nylon and elastane, or ‘cotton-rich’), the quality of construction and the weaving technique.

When wearing GC hosiery, it is essentially the below-knee component that has the greatest effect to compress the superficial venous systems, pushing blood into the deep veins and to enhance the velocity of blood flow seen in the deep veins. A similar compression of the lymphatic system must occur also. The standard profile for GC hosiery (for any class of compression) is such that maximum squeeze is provided at the ankle, reducing to approximately 80% squeeze just below the knee. For a Class I product, this represents approximately 17-21 mm Hg compression at the ankle, reducing to approximately 14 mm Hg below the knee. Such a compression profile acts entirely uniformly around the circumference of the lower limb.

### SUMMARY OF THE INVENTION

The present invention is based on an understanding of the anatomy of the leg, and in particular the perforating (or communicating) veins, i.e. the connections between the superficial and deep venous systems. This invention appreciates the desirability of enhancing the uni-direction of flow of blood from the superficial venous system to the deep veins, and minimising the effects or possibility of reflux of blood from the deep veins outwards into the superficial systems. The former follows what happens under normal circumstances with an intact set of vein valves in the perforating (or ‘“communicating”) veins. The latter is the situation often seen in those with PTS, whereby blood flows via damaged vein valves into the superficial system, abnormally, from the deep system. For everyday use, the regulated, uni-directional flow of blood from the superficial to the deep system might further aid the feeling of general well-being that can be noted with GC hosiery wear and especially also, in those with varicose veins.

According to the present invention, an item of graduated compression hosiery additionally comprises a ‘compression panel’ adapted to provide targeted compression to one or more of the sites for perforating vein communication between the superficial and deeper vein systems in the lower leg.

### BRIEF DESCRIPTION OF THE DRAWINGS

FIG. 1 is a schematic representation, in plan, of the pattern of a compression panel suitable for use in the invention;

FIG. 2 is a schematic view of a sock embodying the invention, when inside out;

FIG. 3 shows the same sock as worn (with pads showing through the material); and

FIG. 4 shows the same sock partly pulled down.

### DESCRIPTION OF THE INVENTION

An item of hosiery according to the present invention may be a conventional GC “sock”, with an added panel. The panel may be of any suitable material, e.g. compressed fluff fibre, silicone, latex rubber, a memory foam, polyurethane foam, or any combination.

The panel may be continuous or, preferably, discontinuous. For example, there may be a regular or random array of pads or protrusions. The protrusions may be built up by depositing a suitable plastics or other material, e.g. by ink-jet printing, or by the application of pre-made transfers. The panel is intended to be in contact with the inside (medial) aspect of the leg. Typically therefore, items of hosiery according to the invention are presented in pairs, with labelling or other indication of their being for use as “left” and “right” socks.

By way of example, the panel may be made up from an array or series of pads, of differing sizes and of differing heights, arranged in such a way as to cover the sites of communicating veins and to provide focused compression on the background of graduated compression. The advantage of having an array of pads like this is also to allow greater movement of the ‘compression zone’ on the underlying garment substrate and to allow some ‘breathability’ between each pad, to help keep the skin from sweating too much. A wicking agent might be included in the garment, to help this same problem. Another optional additive is an anti-bacterial agent, to help prevent general odour.

Each “sock” may or may not have a toe/foot part. It may extend to below or above the knee, or a pair may be presented as tights.

The targeted compression provided by a product of the invention should correspond to the sites of perforating (or communicating) veins. Anatomically, for the vast majority of the population, there are known sites for perforating vein communication between the superficial and deep vein systems in the lower leg. In particular, there are three sites of communication located at approximately 5 cm, 10 cm and 15 cm upwards from the ankle (at the medial malleolus), generally placed towards the postero-medial aspect of the lower calf. The sites can often be felt through the skin as ‘defects’ in the deep fascia and routinely, are located exactly by use of venous duplex ultrasound. Control of the perforating veins at these sites should be advantageous, and should enhance significantly the role of GC hosiery. This aspect of GC hosiery has never been considered previously.

According to this invention, known GC hosiery may simply be modified by the addition of an ‘inside-the-sock’, anatomical-site aware, additional compression panel, designed to give targeted compression to the sites of the perforating veins. Such an additional compression panel would be designed to cover the specific area known commonly to encompass the perforating vein sites and would be constructed so as to be comfortable and smooth against the skin. For example, the panel may be profiled as a smooth, essentially elongate bulge running from the region of the ankle, upwards for approximately 20 cm for example, with a width of approximately one third the circumference of the sock at the ankle part. A suitable material for the panel might be silicone or compressed fluff fibre. One material may be covered by another, separate material and held in place inside the GC product by means of stitching, gluing, heat-welding, or other bonding method. Alternatively, the whole product may be made as one piece by a suitable weaving or knitting means. The panel may be formed or inserted into all types of GC hosiery currently fashioned, e.g., below knee, mid-thigh, full length (leg) and as tights. Furthermore, the panel may be formed or inserted into all classes of compression; low support levels undefined formally, to standard class I, II, III and IV compression level products. The panel may be inserted in products deemed suitable for hospital and retail sector uses, including preventative anti-embolism socks, socks to treat or help manage varicose veins, socks to be worn by those with likely to occur, or established PTS, and those with known lymphatic conditions. Other therapeutic uses might include relief of swollen ankles and lower limbs, as well as use in promoting well-being and enhancing sports recovery and perhaps, performance. In the retail sector, uses include for long-haul travel, for maternity use, for sports use, for military use and for everyday wear.

The invention will now be described by way of example only with reference to the accompanying drawings.

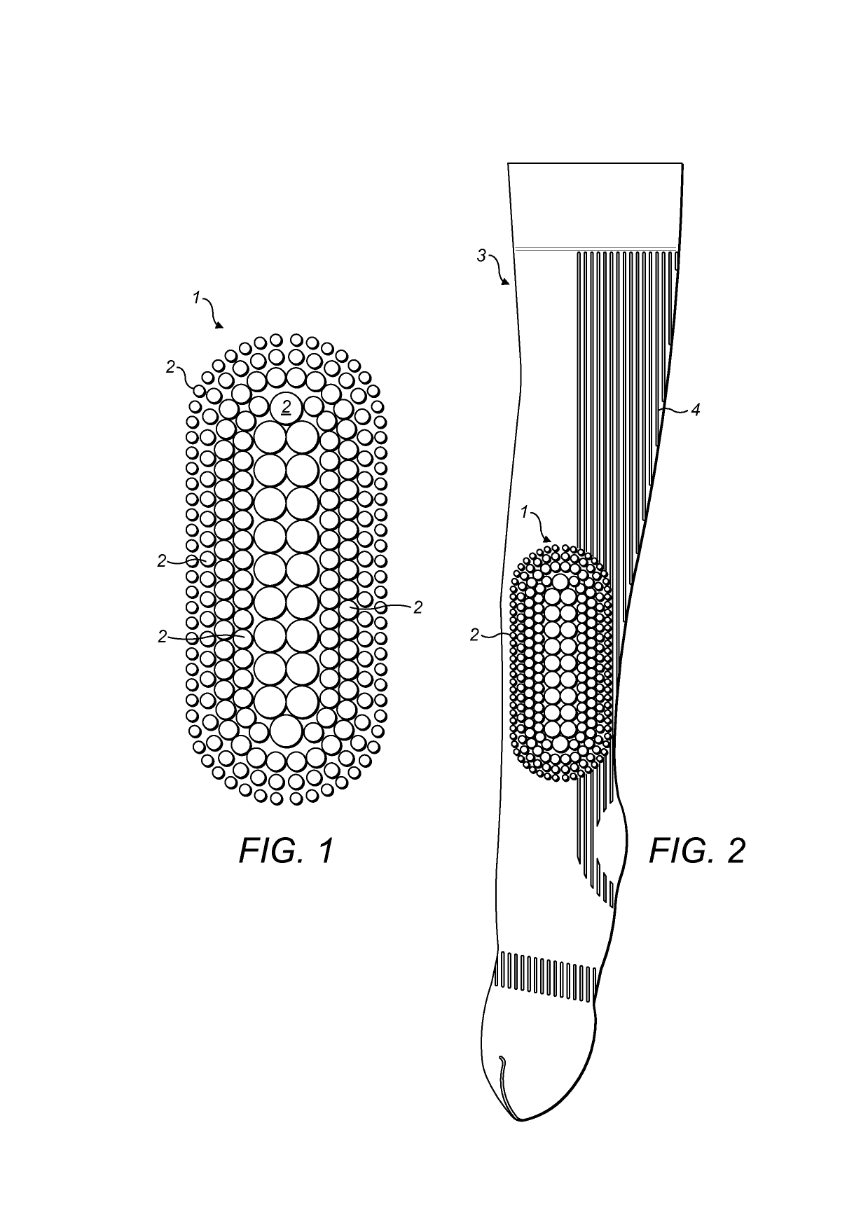

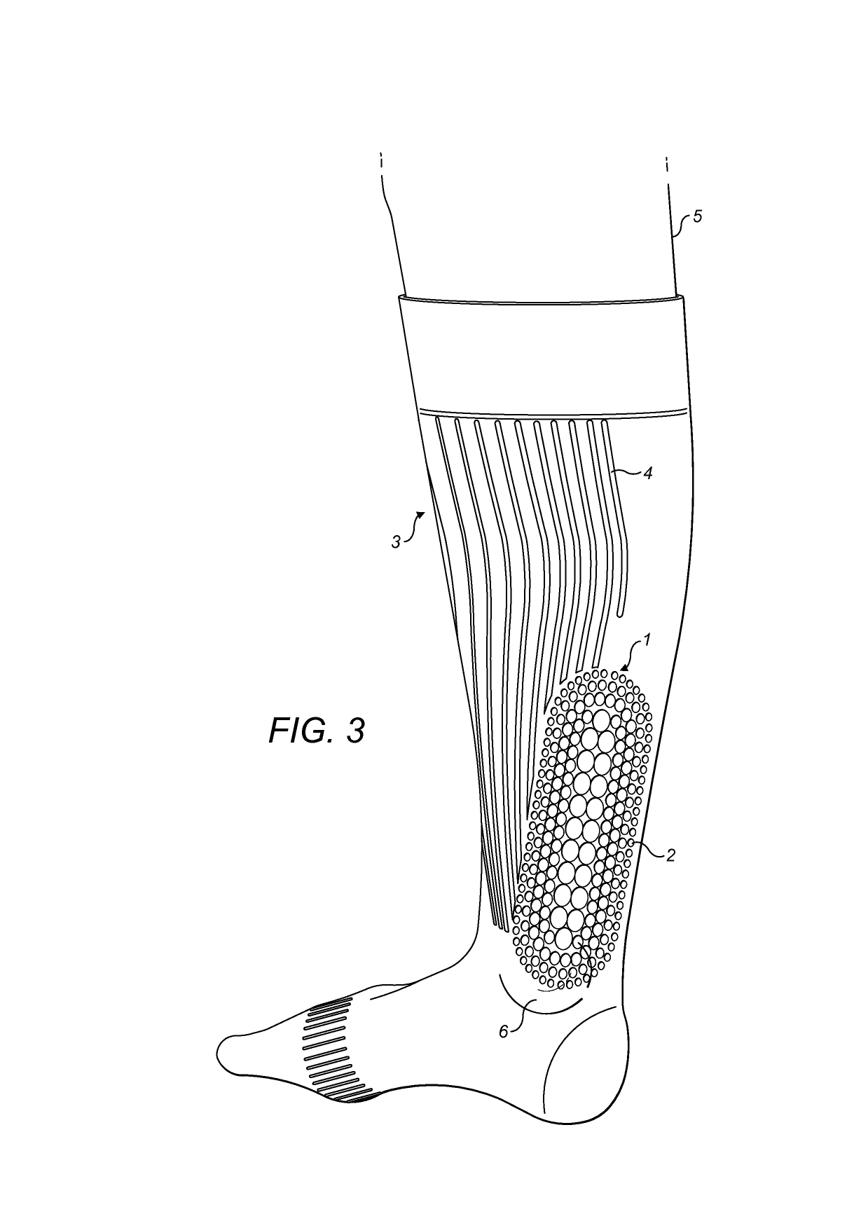

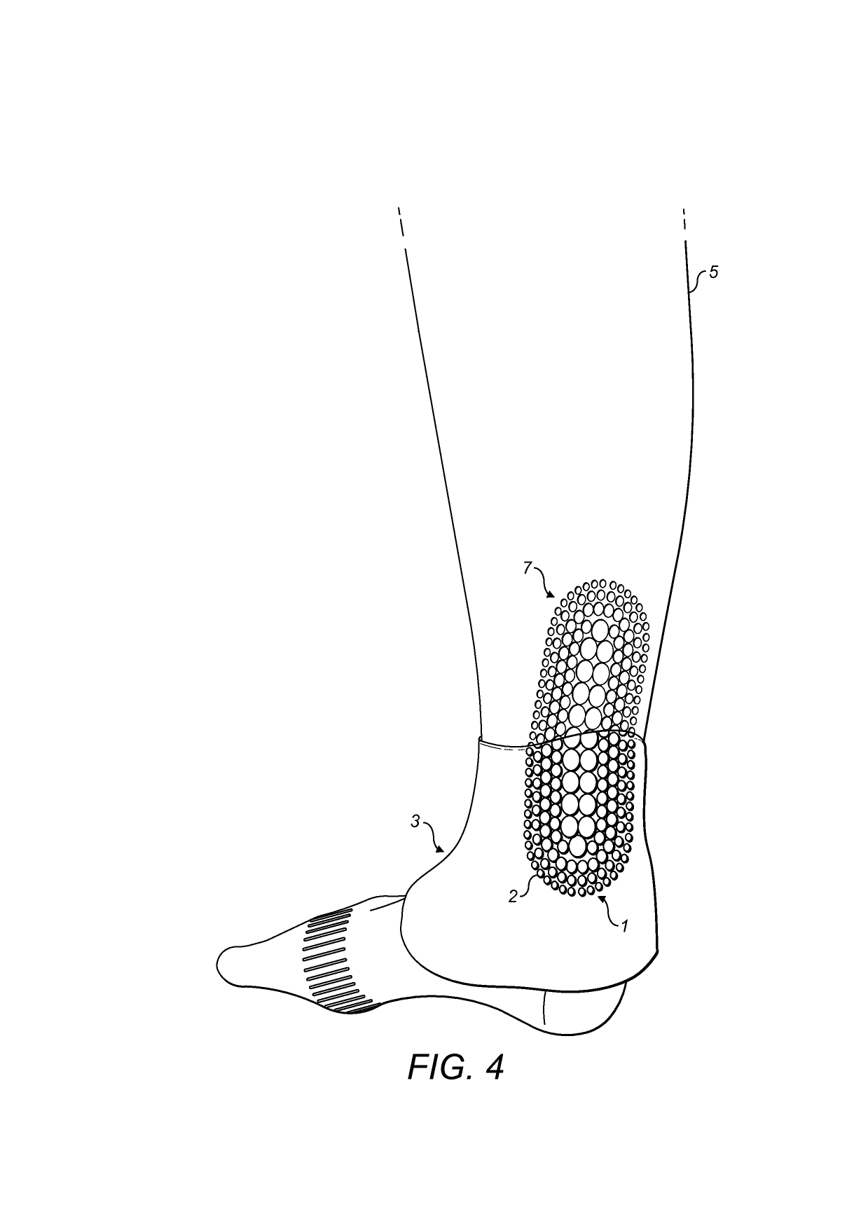

FIG. 1 shows a panel 1 of pads or protruberances 2. In profile, it would be evident that the pads are of different heights, giving a “keel” shape to the panel. FIGS. 2 to 4 show such a panel, in position on the inside of a graduated compression sock, respectively inside out, as worn (with the pads 2 showing through the material of the sock), and partly pulled down; these drawings show a sock 3 of a material 4, a user's leg 5 and ankle 6, and indents 7 left by the pads.

1. An item of graduated compression hosiery comprising a compression panel adapted to provide targeted compression of one or more sites for perforating vein communication between the superficial and deeper vein systems in the lower leg.

2. The item according to claim 1, wherein the or each site is located approximately 50 mm, 100 mm and 150 mm above an ankle at a medial malleolus.

3. The item according to claim 1, wherein the panel is thickest in the middle thereof.

4. The item accordingly to claim 1, wherein the panel is configured as an essentially elongate inward bulge extending upwardly from an ankle.

5. The item according to claim 1, wherein the panel comprises a plurality of protrusions.

6. The item according to claim 1, wherein the panel extends for 150 to 300 mm upwards from ankle.

7. The item according to claim 1, wherein the panel has a width of approximately one-third of the circumference of the item at an ankle and/or the lower leg.

8. The item according to claim 1, for therapeutic use.

9. A method for treating varicose veins wherein said method comprises the use of an item according to claim 1.

10. A pair of items according to claim 1, for use with each panel in contact with the inside of a leg of a wearer.
